(19) United States
(12) Patent Application Publication
(10) Pub. No.: US 2004/0102765 A1
(43) Pub. Date: May 27, 2004

(54) Method for the minimal-to non-invase optical treatment of tissues of the eye and for diagnosis thereof and device for carrying out said method

(76) Inventor: Karsten Koenig, Neuengonna (DE)

Correspondence Address: HAYNES AND BOONE, LLP, 901 MAIN STREET, SUITE 3100, DALLAS, TX, 75202, US

(21) Appl. No.: 10/473,272

(22) PCT Filed: Mar. 26, 2002
(86) PCT No.: PCT/EP02/03370

(30) Foreign Application Priority Data
Mar. 27, 2001 (DE) 101 15 751.7
Sep. 28, 2001 (DE) 101 48 783.5

(51) Int. Cl. A61B 18/20

(52) U.S. Cl. 606/5; 606/11

(57) ABSTRACT

The invention relates to a process for minimally invasive to non-invasive optical treatment of tissues of the eye and also for diagnosis thereof and to a device for implementing this process. The object underlying the invention is to create a process and a laser arrangement for minimally invasive to non-invasive optical treatment in the interior of the eye, particularly of cases of defective vision, by ablation of tissue, said treatment being distinguished by a hitherto unattained high precision, with possible widths of incision in the range less than 2 μm, without a significant mechanical impairment of the surrounding tissue occurring that has been generated by photodisruption. The process and the arrangement are to be inexpensive and easy to operate. In addition, at the same time the arrangement is to enable a three-dimensional imaging of the tissue. This object is achieved by virtue of a process in which the ablation is effected by focused planar or spatial scanning while adhering to equal, in order of magnitude, focusing-point diameters and point spacings below 5 μm with a radiation within the spectral range from 500 nm to 1200 nm, whereby, by virtue of a pulse duration in the order of femtoseconds and an energy of the individual pulse in the order of nanojoules and below, the destruction of the tissue is substantially limited to the diameter of the point, and permanent changes by virtue of propagation of energy beyond this diameter are avoided. The invention can be applied in ophthalmology.

### DESCRIPTION

The invention relates to a process and to an arrangement for minimally invasive to non-invasive ophthalmic surgery by optical treatment of the tissue by means of laser radiation. The process and the arrangement preferably serve for refractive corneal surgery for the treatment of defective vision, in which case “online” diagnosis and monitoring of the therapy may also take place. The arrangement and the process may also be utilised for other surgical procedures in the eye, for example for antiglaucomatous therapy, in order to re-enable regulated drainage of the aqueous humour by laser-induced transection of tissue (boring of a channel) or to reduce the production of aqueous humour through partial removal of the ciliary body. In addition, cysts and tumours and other pathological changes in the tissue on and in the eye can be diagnosed and laser-treated (punctured).

Refractive corneal surgery has conventionally been effected hitherto by invasive mechanical methods, by means of laser radiation or by a combination of mechanical methods with a laser treatment.

In the case of treatment with laser radiation without mechanical methods, typically an excimer laser is employed having a highly absorbent laser wavelength in the ultraviolet (UV) range, with pulse lengths in the nanosecond range. The ablation process is based on so-called photoablation. In the case of treatment with the excimer laser, tissue is ablated from the surface of the cornea, starting at the so-called epithelial layer, to a depth of about 100 μm, in order to obtain a correction of refractive power. One disadvantage is the relatively poor healing as a result of optical removal of the epithelial layer.

On the other hand, in the case of the so-called LASIK process an upper part of the cornea is firstly partially “planed off” with a mechanical device (microkeratome). The partially separated corneal layer, the so-called flap, is folded to one side and exposes the layer of tissue situated underneath with a view to removing tissue. The optical ablation of tissue ensues by means of UV excimer laser. After the laser treatment, the flap is folded back and adheres to the cornea by virtue of adhesive forces. The flattening of the cornea that is produced in this way serves for the correction of short-sightedness. One disadvantage of this treatment is the relatively high proportion (typically 5%) of complications as a result of the initial mechanical intervention. In addition, the flap may slip again, even a long time after the therapy, as a result of mechanical influence, for example vigorous rubbing.

Of particular interest, therefore, is the attempt at minimally invasive to non-invasive optical therapy in the interior of the cornea, particularly in the so-called stroma layer, without injury to the surface of the eye. This can be done, in principle, by focused laser radiation of high intensity having wavelengths in the visible and near-infrared (NIR) wavelength range up to about 1200 nm.

The removal of material is ordinarily effected at extremely high intensities in the range of order of magnitude of GW/cm² and TW/cm² by ionisation of biomolecules as a consequence of non-resonant multi-photon absorption. The first free electrons generated in this way trigger a process which results, via cumulative amplification effects as a consequence of the interaction with the electromagnetic field of the laser radiation and associated absorption of energy by virtue of inverse bremsstrahlung, in laser-induced optical penetration and in formation of plasma. By virtue of the rapid expansion of the plasma, a dynamic high-pressure region arises which brings about the formation of a radially extending shock-wave. The portion of removed material that is caused by shock-waves and formation of bubbles (cavity bubbles, gas bubbles) is designated as photodisruption [Juhasz et al. IEEE Journal of Selected Topics in Quantum Electronics 5 (1999) 902-909]. Ablation fragments can also be transported out of the interactive region by optomechanical means [Loesel et al. Appl. Phys. B 66 (1998) 121-128].

Experiments have been carried out hitherto with nanosecond pulses, picosecond pulses and femtosecond pulses [e.g. Krasnov, Arch. Ophthalmol. 92 (1974) 37-41; Stern et al. Arch. Ophthalmol. 107 (1989) 587-592; Niemz et al. Lasers Light Ophthalmol. 5 (1993) 149-155; Vogel et al. Invest. Light Ophthalmol. 5 (1993) 149-155; Juhasz et al. Lasers Surg. Med. 19 (1996) 23-29].

Nanosecond pulses require high pulse energies and, as a consequence of a high proportion of high mechanical energy, offer only limited therapeutic possibilities in the field of corneal surgery (Steinert and Puliafito, The Nd:YAG laser in ophthalmology, Philadelphia, Pa.; W. B. Saunders, 1985: 11-21). In the case where shorter pulses are used, the threshold for therapeutic penetration falls. Through the use of low-energy pulses, the proportion of destructive mechanical energy can be reduced, as has been demonstrated by the use of picosecond pulses. However, even in this case no optimal treatment has been obtained, this being attributed, in particular, to the formation of bubbles [Niemz et al. Lasers Light Ophthalmol. 5 (1993) 149-155; Gimpel et al. Int. Ophthalmol. Clin. 37 (1997) 95-102; Ito et al. J. Refract. Surg. 12 (1996) 721-728]. Thus the diameter of cavitation bubbles in the case where use is made of nanosecond pulses typically amounts to 1 mm to 2 mm; in the case of picosecond pulses, 0.2 mm to 0.5 mm [Vogel et al. Proc. SPIE 1877 (1993) 312-322]. More favourable therapeutic effects are hoped for through the use of femtosecond pulses.

Previous investigations into refractive corneal surgery with femtosecond pulses have been based on the use of pulses with pulse energies in the microjoule and millijoule range, with pulse repetition frequencies in the Hz to kHz range and laser-illumination spots with a diameter of several micrometres [e.g. Kurtz et al. J. Refract. Surg. 13 (1997) 653-658]. Thus Kurtz et al. describe an arrangement that is characterised by a repetition frequency of 10 Hz, an illumination spot with a diameter of 26 μm, pulse energies up to 10 mJ and variable pulse duration [Kurtz et al., J. Refract. Surg. 13 (1997) 653-658]. Lubatschowski et al. utilised a laser system having a repetition frequency of 1000 Hz, a maximum pulse energy of 1 mJ and an illumination-spot diameter of 7 μm [Graefe's Arch. Clin. Exp. Ophthalmol. 238 (2000) 33-39]. Arrangements of such a type, which typically consist of a laser oscillator and an amplifier and also contain pulse-stretching modules and pulse-compression modules, are space-intensive, care-intensive and cost-intensive.

With these arrangements and laser parameters, incisions in the interior of the cornea having a width of, typically, more than 10 μm can be produced, and material can be ablated in this way. In addition, a flap can be produced by optical means. An appropriate instrument is on the market. In this case, femtosecond pulses having a wavelength of 1053 nm are utilised. The radiation in this case is focused into the eye onto a spot having a diameter of 3 μm and is positioned intraocularly by means of a scanning device. The points of irradiation are situated closely alongside one another in the form of a spiral, with a spatial separation of more than 5 μm, but are temporally offset. Material is removed from the interior as far as the surface of the cornea in such a way that with the aid of a partial vacuum the flap produced by means of laser radiation can be folded to one side. The mechanical production of the flap is thereby dispensed with.

In patents U.S. Pat. No. 5,993,438 and EP 0 903 133 a process for intrastromal photorefractive keratectomy is described which brings about the photodisruption of material in the stroma, whereby the material affected by photodisruption corresponds approximately to the volume of focus with a diameter of, typically, 10 μm to 25 μm and the illumination spots are placed in such a way that their spatial separation corresponds to one to two diameters of the bubbles that are produced and they generate laser-treated layers which are centrosymmetrical relative to the optical axis and which are able to produce a desired cavity in the stroma. In the present invention, a method is described using a pulse repetition frequency within the range from 10 Hz to 100 kHz. Preferred frequencies are 1 kHz to 10 kHz, with an illumination spot having a diameter of approximately 10 μn. The known technical solutions are based on the use of photodisruption, that is to say, the mechanical action of shock-waves and bubbles. The photodisrupted tissue is intended to be absorbed from the cornea or to be transported away out of the cornea.

In patent U.S. Pat. No. 6,146,375 an account is given of the photodisruption of tissue for the treatment of glaucoma with femtosecond and picosecond pulses, partly with the aid of chemical substances that alter the scattering behaviour of the eye.

The relatively high pulse energies used hitherto, in the order of microjoules, which result in undesirable mechanical effects, in particular by virtue of the effect of so-called bubbles and the associated shock-waves as a result of the process of photodisruption, turn out to be a disadvantage of the previous processes by means of femtosecond pulses. Thus an account is given of the formation of bubbles with a size of 25 μm in the case where use is made of 2 μJ pulses with a pulse duration of 300 fs in water, and of coagulations of collagen within the interactive zone [Lubatschowski et al. Graefe's Arch. Clin. Exp. Ophthalmol. 238 (2000) 33-39]. In addition, self-focusing effects which may lead to undesirable damage in the surrounding tissue can be induced at these relatively high pulse energies. The use of these relatively high pulse energies also requires elaborate, cost-intensive and labour-intensive laser systems with amplifiers.

Also a disadvantage is the fact that previous femtosecond laser systems for corneal surgery do not enable high-resolution analysis of the laser treatment. Ordinarily, separate optical systems are utilised for diagnosis (e.g. Arashima et al., EP 0 850 614 A1). In this document a system is described which comprises a laser for corneal ablation, an additional illumination system and a photographic device.

In patent specification U.S. Pat. No. 5,984,916 a process and an arrangement for laser ophthalmic surgery are described which are based on the use of irradiation spots of about 10 μm, pulse frequencies up to 100 kHz and energy densities from 0.2 μJ/μm² to 5 μJ/μm². Such energy densities and pulse frequencies, however, presuppose the use of elaborate laser systems with amplifier, pulse-stretching and pulse-compression units and also pulse energies in the range greater than 0.2 μJ. An integrated diagnostic system is not provided.

The object underlying the invention is therefore to create a process and a laser arrangement for minimally invasive to non-invasive optical treatment in the interior of the eye, particularly of cases of defective vision, by ablation of tissue, said treatment being distinguished by a hitherto unattained high precision, with possible widths of incision in the range less than 2 μm, without a significant mechanical impairment of the surrounding tissue occurring that has been generated by photodisruption and self-focusing. The use of systems that are inexpensive and easy to operate is to be possible. In addition, the same arrangement is to enable a three-dimensional imaging of the tissue for diagnosis, for target analysis, for optical online monitoring of the treatment and for three-dimensional high-resolution optical analysis of the laser treatment.

This object is achieved by virtue of the characterising features of claims 1 and 9. Advantageous configurations are covered by the respectively subordinate claims.

The efficacy of the invention is demonstrated below on the basis of exemplary embodiments, and its functionality is elucidated in greater detail. Shown are:

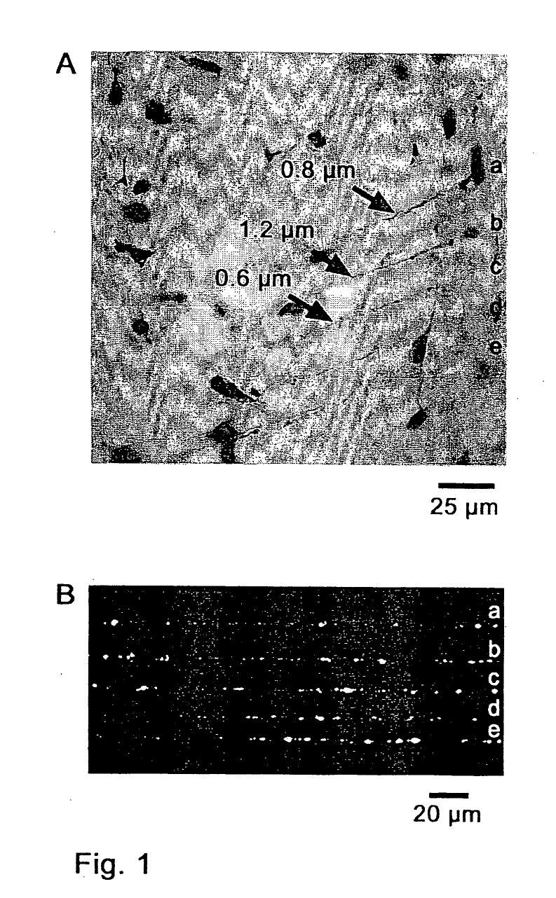

FIG. 1A: HE-stained frozen sections of a region with laser incisions, which provide evidence of the precise cutting in the stroma of a pig's eye with sub-nanojoule, femtosecond laser pulses. A measurement revealed typical widths of incision within the range from 0.3 μm to 1 μm.

FIG. 1B: Reflectance photographs directly after five incisions have been made in the stroma of a pig's eye with, in each case, 20 ms total dwell-time of the beam per pixel and with 512-pixel line scanning.

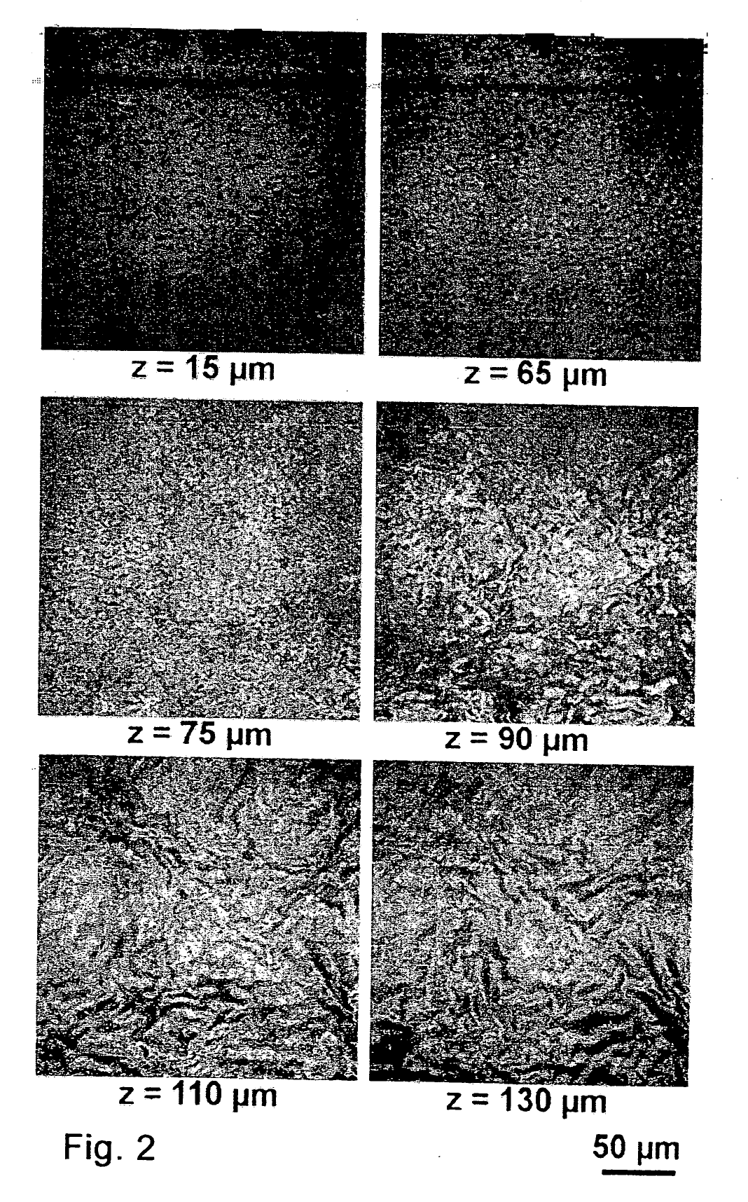

FIG. 2: Photographs of the autofluorescence stimulated with a mean wavelength of 800 nm and Second Harmonic Generation (SHG) with high spatial resolution at various depths of tissue, i.e. in the z-direction, of a pig's eye. The various tissue layers of the cornea and individual cells are clearly discernible.

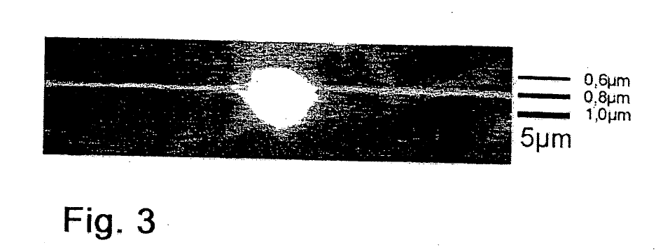

FIG. 3: Fluorescence photograph 2 s after laser therapy has taken place with 2 ms total dwell-time of the beam per pixel. The luminescent region along the incision has a width of about 0.8 μm. The separate, larger luminous area represents the luminescence of a bubble.

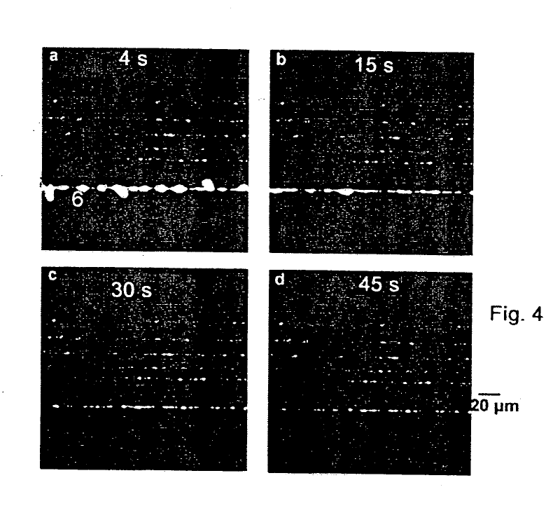

FIG. 4: Reflectance photographs which were taken 4 s, 15 s, 30 s and 45 s after ablation of material with a “Linescan 6” and which yield information about the kinetics of the bubbles. Accordingly, the lifespan of these bubbles lies within the range of less than half a minute.

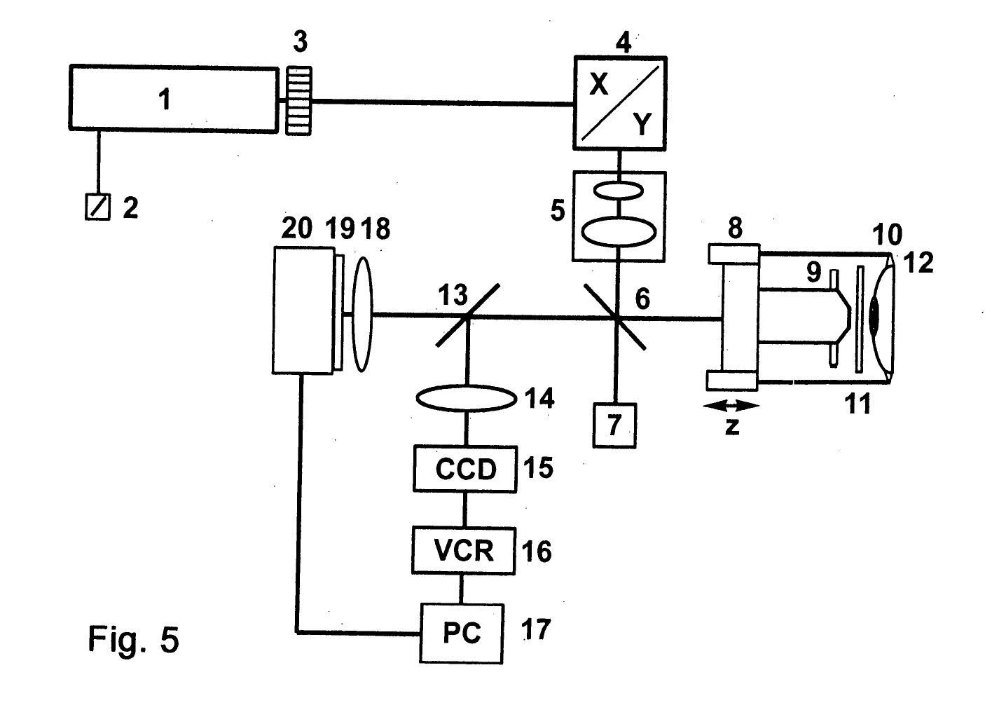

FIG. 5: A schematic representation of an arrangement according to the invention, with a single laser beam.

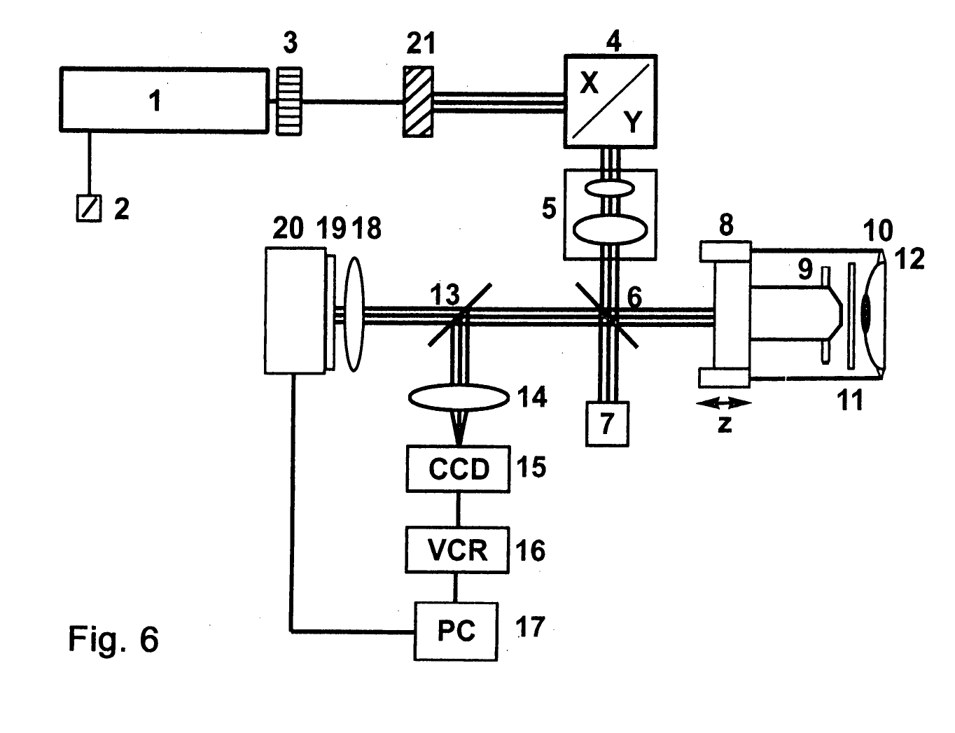

FIG. 6: A representation like FIG. 5 but with a laser beam split up into several single beams.

According to the invention, for minimally invasive to non-invasive optical treatment, for three-dimensional imaging, for optical online monitoring of the treatment and for three-dimensional, high-resolution optical analysis of the laser treatment of tissues of the eye, in particular of the cornea, use is made of focused radiation within the spectral range from 500 nm to 1200 nm, consisting of femtosecond pulses with a pulse energy in the picojoule range and nanojoule range with high repetition frequency in the MHz range and irradiation spots with a diameter less than 5 μm, preferably less than 1 μm, which are moved over the target to be treated, with a typical separation less than 5 μm, as a result of which a precise treatment by selective direct destruction of individual cells or cell constituents or of individual intraocular tissue structures is made possible without irreversible destruction of surrounding areas of tissue, the three-dimensional recording of the tissue to be treated or that has been treated or of individual cells or of individual cell constituents, before and after the laser therapy, is made possible by detection of the fluorescence, preferably of the non-linearly stimulated autofluorescence, or of the reflectance, and also an online monitoring of the therapy is made possible by virtue of spatially and/or temporally resolved online detection of the luminescence of the plasma.

According to the invention, the laser therapy and the three-dimensional imaging of the tissue for the purpose of target analysis, for the purpose of optical online monitoring of the treatment and for the purpose of three-dimensional high-resolution optical analysis of the laser treatment can be realised with only a single arrangement. According to the invention, an arrangement for treatment and for diagnosis comes into operation which consists of a compact femtosecond laser without amplifier within the range from 500 nm to 1200 nm, a beam-guidance system including scanning device, a beam-widener, a high-speed output regulator for switching between diagnosis (target-searching and effect-monitoring) with low-power radiation and therapy with high-power radiation, one or more photon detectors, monitors, beam interrupters, and also suitable automatic control, hardware and software. In order to enable a time-resolved detection of the signals brought about by reflectance, fluorescence and plasma luminescence with a resolution in the picosecond range, according to the invention a high-speed detector, typically a high-speed photomultiplier (PMT), is coupled to a module for time-correlated single-photon counting. For an online observation of effects, a video camera may additionally be employed.

For the focusing of the radiation, use is made of objectives with a numerical aperture greater than 0.8, typically greater than 1.0, and irradiation spots are positioned with a separation less than 5 μm, typically less than 1 μm. For the implementation of the laser therapy, use is made of radiation intensities amounting to more than 100 GW/cm²; for the diagnosis, use is made of lower intensities. The intensities that are variously required are realised by variation of the output of the laser on the specimen. The output regulator has to enable the choice between diagnosis and therapy, and also the adjustment of the light intensity that is required in the given case, depending on the depth of the area of tissue to be investigated or treated.

Surprisingly, in some research it has been found that intraocular ablations of material can be achieved by suitable femtosecond laser pulses in the sub-nanojoule and nanojoule ranges. This became possible through the use of compact laser systems that are easy to operate. The use of elaborate laser systems with amplifier is not required. A hitherto unattainable precision of <1 μm width of incision in the stroma and epithelial tissue was able to be achieved. In this case, individual cells were able to be ablated, individual collagen fibres were able to be separated, or entire regions of tissue were able to be removed, without the surrounding tissue regions being damaged by photodisruption.

In particular, it has become evident that 170-femtosecond pulses having a peak-intensity wavelength of 800 nm, a repetition frequency of 80 MHz in the case where use is made of a focusing optical system with a numerical aperture of 1.3, which enables irradiation spots smaller than 1 μm, at a mean power of 60 mW, corresponding to a pulse energy in the sub-nJ range, make it possible to ablate material in the cornea. The irradiation spot was displaced on the target with a galvanometer scanner. The displacement was effected in steps of less than 1 μm, typically less than 0.5 μm. The temporal interval of a displacement was shorter than 100 μs. The dwell-time of the beam per irradiation spot also lies within the microsecond range, typically within the range less than 10 μs. Each spot was irradiated up to 5000 times, typically around 200 to 500 times. Widths of incision smaller than 1 μm were able to be achieved without damaging surrounding cells of the tissue. These widths of incision were able to be obtained in the epidermis, in Bowman's membrane and in the stroma.

FIG. 1A shows histological HE-stained frozen tissue sections of a pig's eye which demonstrate laser-induced removals of material. Use was made of a mean power of 80 mW. The beam was guided five times along a line (line scan); the dwell-time of the beam per pixel amounted to a total of 20 ms. The width of incision that was achieved varies accordingly from 0.3 μm to approximately 1 μm. No indications of thermal or mechanical damage to the adjacent areas of tissue can be discerned.

FIG. 1B demonstrates reflectance photographs which were taken with the same arrangement directly after implementation of the operations for removal of material. Unexpectedly, on the basis of these photographs it was found that highly reflective zones arose along the cut edges as a result of the laser-induced removals of material. These zones can be imaged three-dimensionally by means of laser radiation of the same wavelength but with substantially lower mean power of less than 1 mW, using suitable photon detectors. The width of these reflecting zones along the incision likewise has values less than 1 μm and therefore correlates approximately with the actual width of incision that can be discerned in the histological image. Interestingly, the bubbles that were generated during the ablation of material also displayed a measurable reflection differing distinctly from the surrounding region. In less strongly reflecting manner, but nevertheless well visible, the 3D reflectance images display distinctly reflecting structures of individual cells in the epithelial layer, in particular the strongly reflecting cell nucleus and the cell membranes, as well as, presumably, collagen structures within the stroma.

Fluorescence photographs were also able to be produced with the same apparatus. At a mean power of 2 mW to 5 mW, a three-dimensional image of the cornea was able to be produced before and after the laser surgery by multi-photon stimulation of endogenous fluorophores in the subfemtolitre volume of focus and by detection of fluorescence with a photomultiplier by scanning of planes at various depths of tissue. In particular, the various tissue layers of the cornea, namely the epithelial layer, Bowman's membrane and the sclera, were able to be clearly located on the basis of the autofluorescence. FIG. 2 shows corresponding photographs of autofluorescence, stimulated at 800 nm, with high spatial resolution at various depths of tissue of a pig's eye.

In particular, by virtue of a two-photon stimulation, the fluorescence of the reduced coenzyme NAD(P)H and also of flavines can be represented. On the basis of the fluorescence, the individual cells can be clearly located. In addition, the collagen fibres of the stroma display a distinct autofluorescence and SHG radiation.

Surprisingly, here too it was found that bubbles arising as a result of the laser treatment can be stimulated by influence of laser light having low power to produce luminescence that is clearly above the intensity of the autofluorescence. In addition, the treated areas along the cutting zone display an autofluorescence that differs from surrounding regions. As a result, the effect of treatment can be made clear with high contrast (FIG. 3).

Interestingly, the plasma luminescence that was produced during the laser irradiation was able to be detected directly with the same photomultiplier during the laser treatment along the treatment area. Thus a statement about the effect of the intense laser radiation is possible in position-resolved manner, and hence an online monitoring of the therapy is provided.

If a wide-field illumination of the target with white light or preferably with light in the near infrared from a halogen lamp or from LEDs is utilised during the laser treatment, the effects of the laser treatment, in particular the formation and the disappearance of bubbles, can be detected online by reflectance measurement, for example with a 50 Hz CCD camera, and, for example, stored on a video recorder or on a PC and reproduced.

By measurement of the reflected and scattered photons and also of the fluorescence photons directly after implementation of the laser therapy, statements can be made on the effect that has been achieved and on the width of incision. In addition, the appearance of bubbles and the dynamic behaviour thereof can be investigated, as FIG. 4 illustrates. Typically, the bubbles arising have dimensions of less than 5 μm and disappear within a few seconds, as represented on the reflected images 4 s, 15 s, 30 s and 45 s after linear ablation of material (6) has taken place.

Since, given suitable pulse energies in the sub-nanojoule range close to the threshold values for optical penetration, ablations of material can be carried out and no indications of mechanical damage to the surrounding region could be found, the ablation of material is possibly not to be ascribed to a photodisruption but merely to a vaporisation of material by virtue of purely thermal effects or by virtue of a photochemical removal of material (breaking-up of bonds by input of energy induced by multi-photon absorption). This assumption is supported by investigations which, close to the threshold value, gave rise to bubbles that do not represent the typical, short-lived cavity bubbles arising as a result of photodisruption [Lubatschowski et al. Graefe's Arch. Clin. Exp. Ophthalmol. 238 (2000) 33-39].

FIG. 5 demonstrates an arrangement according to the invention. By way of source of irradiation for the ablation of material, for the stimulation of the fluorescence and of the luminescence of the bubbles and also of the acquisition of reflectance radiation, a compact femtosecond laser 1 with high repetition frequency with typical values around 80 MHz is employed. The peak-intensity wavelength of the laser lies within the range from 700 nm to 1200 nm; a typical value is 800 nm. The operation of the laser 1 is coupled to a foot-operated switch 2. The laser beam impinges on a high-speed switch 3 with integrated output regulator. This switch is typically an electro-optical switch with switching-times in the microsecond range. It is, in addition, capable of varying the power of the laser and of reducing the initial power of the laser 1 by orders of magnitude. The beam impinges on a scanner 4, which typically consists of two galvanometer mirrors for the x-y deflection. The beam passes across a scanning and widening optical system 5 before it is directed onto the focusing optics 9 via a reflecting mirror 6 acting as a beam-splitter. Typically, the reflecting mirror 6 reflects about 99% of the radiation. The transmitted portions of the radiation, amounting to 1%, impinge on a detector 7 which performs the output measurement and optionally makes a trigger signal available. The focusing optics 9 can be adjusted by means of a piezoelectrically driven adjuster 8 with nanometre precision, and in this way the focal plane can be varied. A mechanical support 11 serves for fixing the position of the eye and is able to receive a glass window 10 which is 170 μm thick. The beam is focused onto the eye 12. Diffusely reflected radiation or radiation that has arisen in the eye 12 is transmitted in a small percentage, typically 1%, through the first beam-splitter 6 and is conducted by a beam-splitting mirror 13 by way of second beam-splitter, on the one hand through an imaging optical system 14 onto a radiation detector 15, typically a CCD camera. The image arising can be recorded, in online and spatially resolved manner, by means of a video recorder 16 and a personal computer 17. Luminescence radiation is conducted by the beam-splitters 6 and 13, the one optical system 18 and a filter 19 onto a radiation detector 20. This radiation detector 20 detects the fluorescence, the luminescence of the plasma and the luminescence of the bubbles. According to the invention, this radiation detector 20 may be a photomultiplier (PMT) with conventional response-time, a high-speed PMT in conjunction with a Single Photon Counting (SPC) module with time resolution in the picosecond range, or a spectrometer with photon detector, typically a polychromator and a CCD camera.

The signal is edited by suitable image processing in the personal computer 17 so as to form clear planar and spatial images, depending on the position of the scanner 4 and optionally taking account of the signal of the detector 7.

If the optical system 18 is constituted by a suitable imaging optical system, CCD cameras may also act as detectors.

In addition, a module 21, as represented in FIG. 6, may be integrated which, instead of the scanning process with only one beam, also enables simultaneous or virtually simultaneous scanning with several beams. Such a module 21 may typically be integrated into the beam path of the laser between the switch 3 and the scanner 4. This module may include known multi-lens arrangements or beam-splitters. A temporal offset of the component beams in the femtosecond and picosecond range is likewise possible. The distribution of the component beams in the target may in this case favourably be a matrix in the form of a rectangular area or circular area or in the form of a line. In the module 21, or inserted in the beam path upstream or downstream of said module, an output regulator which is preferably effective as a reducer may be arranged, in order to lower the continuous laser radiation, in accordance with the invention, from the “treatment level” to the “diagnosis level”.

List of Reference Symbols

1 laser

2 foot-operated switch

3 switch

4 x-y deflection system

5 widening optics

6 first beam-splitter

7 detector for output measurement and control

8 z-direction fine adjustment

9 focusing optics

10 glass window

11 mechanical support

12 eye

13 second beam-splitter

14 imaging optics

15 radiation detector for reflectance measurement

16 video recorder

17 personal computer

18 optics

19 filter

20 radiation detector for secondary radiation

21 module for splitting and optionally temporally offsetting the laser beam

1. A process for minimally invasive to non-invasive optical treatment and recognition of tissues of the eye by means of pulsed laser radiation, in particular for refractive corneal surgery, characterised by focused linear, planar or spatial scanning while adhering to equal, in order of magnitude, focusing-point diameters and point spacings below 5 μm with a radiation within the spectral range from 500 nm to 1200 nm, wherein, by virtue of a pulse duration in the order of femtoseconds and an energy of the individual pulse in the order of nanojoules and below, the destruction of the tissue is substantially limited to a region <5 μm around the focusing-point and permanent changes by virtue of propagation of energy beyond this region are avoided.

2. Process according to claim 1, characterised in that the desired ablation power is obtained by multiple pulse influence in the region of the same focusing-point.

3. Process according to one of the preceding claims, characterised in that pulse repetition frequencies in the MHz range are employed.

4. Process according to one of the preceding claims, characterised in that a radiation with a point diameter from 0.3 μm to 1 μm, with a peak-intensity wavelength of 800 nm, a pulse duration of less than 300 fs, an energy of the individual pulse of <10 nJ and also a pulse repetition frequency of 80 MHz is employed.

5. Process according to one of the preceding claims, characterised in that the reflectance radiation and/or the secondary radiation of optical effects, such as, for example, non-linearly stimulated autofluorescence or plasma luminescence, are evaluated during and after the treatment with a view to monitoring the therapy.

6. Process according to one of the preceding claims, characterised in that by using the otherwise identical laser pulses, but with reduced power, reflectance radiation and/or secondary radiation is/are generated and evaluated with a view to diagnosis and also with a view to monitoring the therapy.

7. Process according to claim 6, characterised in that with a view to realising an online monitoring of the therapy the emission of treatment pulses and pulses of reduced power is effected in alternation.

8. Process according to claim 6 and 7, characterised in that the mean power of the laser for diagnosis and monitoring of the therapy is lowered to −0.1% to 10% of that for the purpose of treating the tissue.

9. An arrangement for minimally invasive to non-invasive optical treatment and recognition of tissues of the eye, in particular for refractive corneal surgery, with a pulsed laser and a device for focusing the laser radiation in a linear, planar or spatial pattern, characterised in that the treatment-beam path extends from the laser (1) via a high-speed, preferably electro-optical switch (3), an x-y deflection system (4), a widening optical system (5), a first beam-splitter (6) and a focusing optical system (9) with a z-direction fine adjustment (8) to the eye (12) of the patient, the first beam-splitter (6) being transparent to a fraction of the radiation conducted to the eye (12) in the direction of a detector (7) for the purpose of output measurement and control and also to the radiation coming from the eye (12) in the direction of an evaluation-beam path.

10. Arrangement according to claim 9, characterised in that the switch (3) is an output regulator at the same time or in that an output regulator is arranged upstream or downstream of the switch (3).

11. Arrangement according to claim 9 or 10, characterised by a second beam-splitter (13) in the evaluation-beam path for the purpose of splitting up the reflectance radiation and the secondary radiation to radiation detectors (15 and 20, respectively) that are specific for the given radiation.

12. Arrangement according to claim 11, characterised in that the outputs of the radiation detectors (15 and 20) are connected to common evaluation (17) and display (16) devices.

13. Arrangement according to claim 12, characterised in that the output of the detector (7) for output measurement and control is also connected to the common evaluation (17) and display (16) devices.

14. Arrangement according to one of claims 11 to 13, characterised in that the radiation detector (20) for the secondary radiation is a photomultiplier.

15. Arrangement according to one of claims 11 to 13, characterised in that the radiation detector (20) for the secondary radiation is a high-speed photomultiplier in conjunction with a single-photon detector with a time resolution in the order of picoseconds.

16. Arrangement according to one of claims 11 to 13, characterised in that the radiation detector (20) for the secondary radiation is a spectrometer with photon detector.

17. Arrangement according to claim 16, characterised in that the radiation detector (20) for the secondary radiation is a polychromator in conjunction with a CCD camera.

18. Arrangement according to one of claims 9 to 17, characterised by a module (21) arranged between the switch (3) and the x-y deflection system (4) for the purpose of splitting up the laser beam into several spatially offset single beams.

19. Arrangement according to claim 18, characterised in that the module (21) is also suitable for temporally offsetting the single beams in the order of femtoseconds to picoseconds.
